(19) United States
(12) Patent Application Publication
(10) Pub. No.: US 2025/0346615 A1
(43) Pub. Date: Nov. 13, 2025

(54) GLYCEROPHOSPHOINOSITOL IN PREVENTING AND TREATING COVID-19 INFECTIONS AND METHOD FOR OBTAINING IT

(71) Applicant: PLANTAREI BIOTECH S.R.L., Padova (IT)

(72) Inventors: Elena SGARAVATTI, Padova (IT); Barbara ZAVAN, Padova (IT)

(21) Appl. No.: 18/866,388

(22) PCT Filed: May 16, 2023
(86) PCT No.: PCT/IB2023/055005

§ 371 Date: Nov. 15, 2024

(30) Foreign Application Priority Data
May 17, 2022 (IT) 102022000010199

(51) Int. Cl. C07F 9/117; A61K 9/00; A61K 9/20; A61K 31/683; C12P 7/62

(52) U.S. Cl. CPC C07F 9/117; A61K 9/0019; A61K 9/2054; A61K 31/683; C12P 7/62

(57) ABSTRACT

Glycerophosphoinositol (GPI) is used in preventing and treating COVID-19 infections. An environmentally sustainable method is for obtaining glycerophosphoinositol. In particular, a process for preparing glycerophosphoinositol from crude or partially purified phospholipid mixtures, includes in sequence: a) hydrolysis of a crude or partially purified phospholipid mixture by treatment with PLA1 and PLA2 enzymes; b) microfiltration of the mixture from step a) and subsequent ultrafiltration and nanofiltration of the microfiltrate to give a concentrated aqueous fraction of reaction products; c) electrodialysis of the aqueous fraction of step b) for separating ionic compounds from neutral compounds; d) ion exchange chromatography. Moreover, glycerophosphoinositol is used in preventing and treating a COVID-19 syndrome.

### CROSS-REFERENCE TO RELATED APPLICATIONS

The present application is a National Stage Filing of PCT International Application No. PCT/IB2023/055005 filed on May 16, 2023, which claims priority to Italian application No. 102022000010199, filed with the Italian Patent Office on May 17, 2022, which applications are incorporated herein by reference in their entirety.

### TECHNICAL FIELD OF THE INVENTION

The present invention relates to the use of glycerophosphoinositol (GPI) in treating and preventing COVID-19 infections and similar viruses, and a method for obtaining it.

### BACKGROUND ART

In mammals, the mobilization of arachidonic acid from the sn2 ester bond of phospholipids is largely due to the activation of cytosolic phospholipase A2 (cPLA2) following receptor activation induced by a number of agonists including hormones, neurotransmitters, neuropeptides, and growth factors.

A substance generated in conjunction with the release of arachidonic acid, i.e., L-α-glycero-phospho-D-myo-inositol (GPI), is an autacoid and is known for the negative cPLA2 modulating properties thereof, thus as an anti-inflammatory substance.

The following structure formula (I) shows the choline salt of GPI by way of explanation:

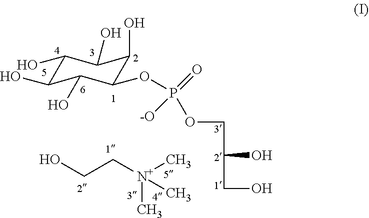

As shown in FIG. 1, cPLA2 initially hydrolyzes phosphatidylinositol (PI) into arachidonic acid (AA) and lyso-phosphatidylinositol; the latter is also a substrate for another enzyme (lysophospholipase A1), resulting in the release of sn-1-linked fatty acid (e.g., stearic acid, SA) and GPI.

GPI negatively modulates cPLA2 through a physiological feedback mechanism, thereby inhibiting the inflammatory process. In fact, an increase in the endogenous level of AA, due to intense or prolonged pro-inflammatory stimuli, induces an increase in the level of GPI that negatively modulates cPLA2 activity. GPI inhibits inflammation at the same level as cortisone agents, but as mentioned the mechanism of action is completely different, whereby it does not induce the typical side effects of cortisone agents, as it does not interfere with the hormonal balance of the body and therefore has no immunosuppressive effect.

Sepsis syndrome commonly occurs in response to lipopolysaccharides (LPS; also known as endotoxins) from Gram-negative bacteria. Tissue factor (TF) is the high affinity receptor and cofactor for factor (F) VII/VIIa. The TF-FVIIa complex is the main initiator of blood clotting and plays an essential role in hemostasis. TF is expressed on perivascular cells and epithelial cells on organ and body surfaces, where it forms a hemostatic barrier. TF also provides additional hemostatic protection to vital organs, such as the brain, lungs, and heart. Under pathological conditions, TF can trigger both arterial and venous thrombosis. In sepsis, the inducible expression of TF on monocytes leads to disseminated intravascular coagulation.

TNFα and IL-1 are the prototypical inflammatory cytokines that mediate many of the cellular events related to LPS exposure. They are released rapidly (30-90 minutes after the inflammatory stimulus) after exposure to LPS and in turn amplify the inflammatory response. Furthermore, many other cytokines, including IL-1 and IL-6, are potent clotting inducers. Coagulation disorders are common in sepsis and in a number of cases (30-50% of patients) they also evolve into a more severe clinical form, i.e., disseminated intravascular coagulation. In sepsis, LPS (or other bacterial components) initiate the coagulation cascade through the induction of tissue factor (TF) expression on mononuclear and endothelial cells. TF in turn activates a proteolytic cascade (coagulation cascade), which eventually leads to the conversion of prothrombin to thrombin and the consequent activation of fibrin. The net result of this sequence of events is the deposition of fibrin clots in the small blood vessels resulting in reduced tissue perfusion, multiple organ failure and, in several cases, death of the subject.

Sepsis is the most common complication seen in COVID-19 patients. As reported in a study of a cohort of 191 patients, more than half of them developed sepsis. It has been seen that the molecular mechanism underlying the observed increase in inflammation by the spike (S) protein of SARS-CoV-2 depends on specific and distinct interactions between the S protein and LPS, leading to changes in the biophysical state of LPS. It can be assumed that sepsis is directly caused by SARS-CoV-2 infection. Comorbidities such as obesity, type 2 diabetes (T2D), cardiovascular disease (CVD), the advanced age of patients and the ethnicity thereof are recognized as important factors for worsening the outcome of the disease: all these comorbidities have something in common and are linked through virus-bacterial interactions, initiated by the translocation of bacterial products, such as lipopolysaccharide (LPS), from the intestine to circulation. In fact, increased plasma levels of LPS and LPS binding protein (LBP) are found in obesity and diabetes, and intestinal dysbiosis is involved in the pathogenesis of insulin resistance. Low-level inflammation induced by the systemic prevalence of bacterial products is involved in vascular abnormalities and circulating LPS levels are significantly modified in CVD.

Given the incidence of the aforesaid complications in the most serious cases of COVID-19 patients, there is therefore a need to provide a preventive and/or curative treatment of SARS-Cov-2 infection.

The preparation of the salts of L-α-glycerylphosphoryl-D-myoinositol (GPI) by chemical synthesis dates back to the patent to Tronconi, G. (U.S. Ser. No. 00/530,6840A) and the literature cited therein; the process begins with a basic transesterification reaction for Na or K alcoholate in alcoholic environment; such a reaction has a yield of about 50-60% in GPI. This reaction, conducted in an alcoholic environment, already allows a partial separation of the GPI salts, which remain insoluble in alcohol with respect to triglycerides, fatty acids and other derivatives of the reaction which are instead soluble in alcohol. Such a patent claims a purification method based on chromatography on weak base resin and subsequent selective crystallization.

In U.S. Pat. No. 6,924,130 B1, Barenholz, Y and Amselem, S. claim enzymatic transesterification or hydrolysis of phospholipids conducted in aqueous environment by using silica particles, which forms a solid/liquid interface offering high conversion rates. This patent focuses on the application of PLC and PLD, also mentioning the use of PLA1 and PLA2.

In CN106459107A the authors report a method for purifying L-α-glycerophosphorylcholine with the use of DMSO, ethanol, methanol and isopropanol solvent after a purification process in ion exchange resin chromatography.

### SUMMARY OF THE INVENTION

Therefore, the present invention first relates to glycerophosphoinositol (GPI) for use in preventing and treating a COVID-19 syndrome.

In particular, the invention is directed to a process for preparing glycerophosphoinositol from crude or partially purified phospholipid mixtures, comprising the following steps in sequence:
a) hydrolysis of a crude or partially purified phospholipid mixture by treatment with PLA1 and PLA2 enzymes;
b) microfiltration of the mixture from step a) and subsequent ultrafiltration and nanofiltration of the microfiltrate to give a concentrated aqueous fraction of reaction products;
c) electrodialysis of the aqueous fraction of step b) for separating ionic compounds from neutral compounds;
d) ion exchange chromatography

The invention further relates to glycerophosphoinositol (GPI) for use in the prevention and early treatment of a COVID-19 syndrome, in particular COVID-19 sepsis, or to the use of GPI for the preparation of a medicament for the prevention and early treatment of a COVID-19 syndrome, or a method for preventing or early treating a syndrome due to COVID-19 or other viral infections, comprising administering an effective amount of glycerophosphoinositol.

These and further objects, as outlined in the appended claims, will be described in the following description. The text of the claims should be considered included in the description in order to assess the description sufficiency.

Further features and advantages of the invention will become apparent from the following description of preferred embodiments, given by way of non-limiting indication.

### BRIEF DESCRIPTION OF THE DRAWINGS

FIG. 1 shows enzymatic hydrolysis from phosphatidylinositol to give glycerophosphoinositol;

FIG. 2 shows the general structure of phospholipid molecules;

FIG. 3 shows a graph reporting the protein expression level expressed as fold increase over stimulated cells treated with LPS stimulus and with GPI, after 5 hours treatment;

FIG. 4 shows a graph reporting the protein expression level expressed as fold increase over stimulated cells treated with LPS stimulus and with GPI, after 24 hours treatment;

FIG. 5 shows a graph reporting the miRNA expression related to M1 (inflammatory, left) or M2 (antiinflammatory, right) commitment of Macrophages after 24 H of treatment;

FIG. 6 shows a graph reporting the production of IL12 and 23 during inflammation expressed as fold increase over stimulated cells treated with LPS stimulus and with GPI, after 24 hours treatment.

### DETAILED DESCRIPTION OF THE INVENTION

In a first aspect, the present invention is directed to the process for preparing L-α-glycerylphosphoryl-D-myoinositol (hereinafter also referred to as glycerophosphoinositol or GPI) from crude or partially purified phospholipid mixtures and for the parallel production of concentrated and purified solutions of L-α-glycerylphosphoryl-D-choline and L-α-glycerylphosphoryl-D-ethanolamine.

The term “mixtures of crude or partially purified phospholipids” means crude lecithins, whether of vegetable or animal origin, also containing triglycerides, purified lecithins containing only phospholipids and further purified lecithins containing only the precursor phosphatidylinositol.

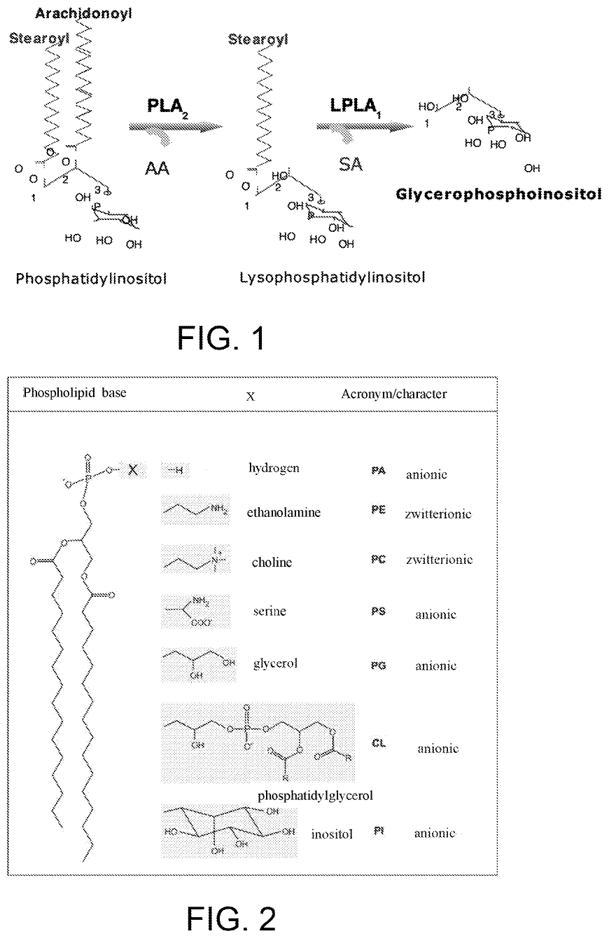

FIG. 1 shows the scheme of enzymatic hydrolysis starting from phosphatidylinositol to give glycerophosphoinositol, while FIG. 2 shows the general structure of some phospholipid molecules which by deacylation can give rise to the respective glyceryl derivatives. Particularly interesting for the purposes of the present invention are the molecules indicated by the abbreviations PI, PE and PC which, by deacylation, respectively give L-α-glycerylphosphoryl-D-myoinositol (GPI), L-α-glycerylphosphoryl-D-ethanolamine (GPE) and L-α-glycerylphosphoryl-D-choline (GPC).

The process according to the present invention comprises the following steps in sequence:
a) hydrolysis of a crude or partially purified phospholipid mixture by treatment with PLA1 and PLA2 enzymes;
b) microfiltration of the mixture from step a) and subsequent ultrafiltration and nanofiltration of the microfiltrate to give a concentrated aqueous fraction of reaction products;
c) electrodialysis of the aqueous fraction of step b) for separating ionic compounds from neutral compounds;
d) ion exchange chromatography.

Step a) of enzymatic hydrolysis is carried out on a reaction mixture with a phospholipid concentration in water between 10 and 100 g/kg of mixture, preferably between 20 and 80 g/kg of mixture, even more preferably between 40 and 60 g/kg of mixture. In fact, enzymatic hydrolysis requires that the interaction between the enzyme and the phospholipids themselves be maximum, which, by forming micelles, reduce the interaction between the reaction site of the enzyme and the substrate itself. Operating with the above concentrations results in the formation of micelles which are not very stable and therefore more easily attackable by enzymes.

In a particularly preferred embodiment, step a) of enzymatic hydrolysis is conducted on a fine dispersion of lecithin in water in which the lecithin has a concentration between 45 and 55 g/kg, or about 50 g/kg.

The amount of enzyme (PLA1 and PLA2) is in the range between 200 and 4000 mg/kg of mixture, depending on the reaction temperature and the overall reaction end time.

The enzymes PLA1 and PLA2 can be used in two consecutive steps, operating first with PLA2 and then with PLA1 as shown in FIG. 1, or in a single step.

In a preferred embodiment, by operating with a mixture of PLA1 and PLA2, the concentration of PLA1 and the concentration of PLA2 are between 800 and 1,300 mg/kg of aqueous suspension, still more preferably between 950 and 1050 mg/kg of aqueous suspension, respectively.

The enzymatic hydrolysis reaction is carried out at a pH between 3.5 and 5.5 or between 3.5 and 5 and at a temperature between 15° C. and 55° C. or between 15° C. and 45° C., preferably between 25° C. and 40° C. The reaction mixture, consisting of a suspension, is initially at a pH about 6.5, but is acidified in the above range (pH=3.5-5) preferably by addition of concentrated phosphoric acid. The pH of the solution and the reaction temperature must be carefully maintained in the above ranges, as competitive hydrolysis reactions of phosphate-glycerol bonds or phosphate-X radical bonds can significantly affect the reaction yield. In a particularly preferred embodiment, operating with a mixture of PLA1 and PLA2, the reaction temperature is between 28° C. and 32° C., or about 30° C., and the pH is between 3.8 and 4.2.

In step a), the conversion yield from phospholipid to glyceryl derivative is between 60% and 90%, depending on the starting phospholipid, with an average value generally between 75% and 80%, or about 78%.

Step b) includes first adjusting the pH to about 7, for example by adding concentrated aqueous solutions of sodium or potassium hydroxide. The reaction mixture is then subjected to microfiltration and subsequent ultrafiltration, at said pH of about 7.

The microfiltration is carried out in tangential filtration on polymer membranes, ceramic membranes or steel membranes, as the phospholipid matrix used and the concentration factors of the recirculating solution to be obtained vary.

The steel membranes used are sintered membranes, consisting of a porous metal core of 316L stainless steel with walls of small thickness (0.5-1.0 mm), the inner diameter of which is covered by a thin titanium oxides layer with a very small pore size (up to 20 nm). These features allow using metal membranes in microfiltration and ultrafiltration systems for the food (soft drinks, dairy products, etc.), pharmaceutical, chemical and petrochemical industries or wastewater treatment, among other applications. In this particular application, a membrane with porosity of about 0.1 micron is preferably used.

Ceramic membranes can also be used equally in this process for microfiltration, preferably using membranes of 0.1 micron porosity.

The ceramic membranes have a support of mixed oxides of titanium, zirconium and aluminum and active layer of zirconium oxide or titanium oxide, are built with the purest materials, and are available in a range of porosity ranging from narrow ultrafiltration to wide microfiltration. The standard porosities for microfiltration are: 0.14-0.2-0.45-0.8-1.4 microns.

Tubular ceramic membranes can be used in highly harsh conditions, maximum temperature=350° C., maximum pressure=10 bar, washing with NaOH (pH=14) at 85° C., washing with HNO₃ (pH=0) at 50° C., hot sterilization (water or steam). There are many configurations available, with the possibility of varying the number and diameter of the channels and the length of the element.

All the membranes described above are commercially available.

The porosity of the membrane can range from 0.45 to 0.1 microns. Filtration, followed by diafiltration to maximize product recovery, preferably occurs with ceramic or steel membranes with porosity of about 0.1 micron.

The microfiltration aims to separate the water-soluble component from the amphiphilic substances still present which tend to form micelles.

The subsequent ultrafiltration and nanofiltration processes are functional for the removal or selection of classes of substances based on the molecular weight thereof and the size thereof in solution. The first ultrafiltration process serves to eliminate enzymes and fractions of other high molecular weight molecules, such as residual proteins present in the raw material, which remain in recirculation, while the other components pass into the permeate. The subsequent nanofiltration is functional for the removal of fatty acids which, at acidic pH, are eluted in the permeate while other molecules, including GPI, GPC, GPE remain in recirculation. The first ultrafiltration process is made using membranes with a cut-off in the range from 20 kDa to 500 kDa, preferably from 30 kDa to 150 kDa, more preferably from 40 kDa to 80 kDa.

Preferably, a second ultrafiltration process can be made at a lower cut-off in order to remove other polymers and oligomers having an average molecular weight, that are kept in recirculation, while GPI, GPC and GPE are collected in the permeate. The subsequent nanofiltration process removes the fatty acids that, at acidic pH, are eluted in the permeate, while GPI, GPC, GPE as well as other molecules are kept in recirculation. The second ultrafiltration process is made using membranes with a cut-off in the range from 1 kDa to 10 kDa, preferably from 2 kDa to 8 kDa, more preferably from 4 kDa to 6 kDa.

Nanofiltration is carried out on wound spiral polymer membranes, with a cut-off value ranging from 150 Da to 3000 Da, preferably from 150 Da to 1000 Da, more preferably from 200 Da to 800 Da or from 150 Da to 300 Da.

In a preferred embodiment, step b) comprises the following sub-steps:
b1) ultrafiltration of the microfiltration product with a wound spiral polymer PES membrane with a cut-off value of about 50,000 Da;
b2) subsequent ultrafiltration with a membrane having a cut-off of about 5000 Da, wherein GPI, GPE, GPC and other molecules with low molecular weight permeate and further oligomers recirculate;
b3) membrane nanofiltration with a cut-off value of about 1000-150 Da, with a wound spiral PES membrane, thus obtaining a partial separation between GPI, kept under recirculation, and GPC and GPE passing with the permeate;
b4) diafiltration with demineralized water with low conductivity so as to obtain a recirculation solution mainly containing GPI and relatively lower concentrations of GPC and GPE with respect to the fraction of step b3).

The GPI-enriched fraction, but with the presence of GPC and GPE, has the GPI component charged and the GPC and GPE components predominantly neutral (internal salts).

Step c) of electrodialysis is functional for the separation of charged molecules from amphoteric molecules or of charged molecules but with relatively high molecular weight and poor ion mobility from inorganic ions with low molecular weight and high ion mobility, if the separation via tangential microfiltration was not sufficiently efficient. The aqueous solution is circulated through an electrodialysis system, thus obtaining the migration of the GPI salts and the preservation of GPC and GPE in the main flow solution.

In preferred embodiments, the permeated solution is further subjected to a second stage of nanofiltration with membranes in the range from 1000 Da to 150 Da to eliminate mainly inorganic substances with low molecular weight and to concentrate the solution.

Step d) of ion exchange chromatography allows obtaining purities >90% especially in the production of GPI, but also of the other glycerophospholipids (GPE and GPC).

The ion exchange chromatography comprises i) the preliminary ion exchange on strong cationic resin in H+ form of the aqueous solution containing GPI, GPE and GPC at varying concentrations and then ii) the passage of the eluted solution on anionic resin in strong or weak OH— form, depending on the impurities to be removed.

For step i), the resin to be used is a strong cationic resin, usually a polystyrene/divinylbenzene gel type resin having a sulfonic group as a functional exchange group and being conditioned in acid form H⁺; the loading flow rate of the solution is preferably about 1.5 BV/h (bed volume per hour), after regenerating and conditioning it with 2 BV of 1M HCl at a flow rate of 1.5-2 BV/h.

For step ii), the resin to be used is a weak anionic macroreticular resin, consisting of a styrene/divinylbenzene copolymer support functionalized through a tertiary amine and conditioned in OH⁻ form; the loading flow rate of the solution is preferably about 1.5 BV/h (bed volume per hour), after regenerating and conditioning it with 2 BV of 1M NaOH at a flow rate of 1.5-2 BV/h.

If using weak anionic resins, the resin is first charged with the solution from the acid exchange, then washed with water, with a diluted solution of formic, acetic or propionic acid at a concentration of about 1-3% w/v to remove the less acidic impurities. The resin is then eluted with a 5-10% w/v formic, acetic or propionic acid solution or with an ammonium carbonate solution at a concentration of 0.1-0.5 g/l, to obtain a pure GPI solution.

The purification of GPE and GPC from the relevant mixture (also containing traces of GPI) can be achieved by using passages on strong cationic resin and strong anionic resin.

In a particularly preferred embodiment, step d) is carried out with a strong sulfonic resin in H+ form, followed by a weak resin in OH— form, washing the weak base resin first with acetic acid 12 g/l, and then eluting it with ammonium carbonate at a concentration of 0.03 M.

In a second aspect, the present invention is directed to glycerophosphoinositol (GPI) for use in preventing and treating a COVID-19 syndrome.

In particular, the invention is directed to glycerophosphomyoinositol (GPI) for use in the prevention and early treatment of COVID-19 sepsis.

GPE and GPC can be used as drugs, supplements or dietary products for example for the stimulation of neurological functions, such as memory stimulation or even the treatment of involutional brain syndromes of the elderly.

### Experimental Section

### Example 1

Enzymatic Hydrolysis of Phospholipids from Defatted Sunflower Lecithin

10 g of defatted sunflower lecithin are finely dispersed with rotor/stator stirrer in 200 g of water for 5 minutes; enzymes PLA1 and PLA2 are then added at a respective concentration of 1000 mg/kg and the suspension is left to react under stirring for 24 hours at a temperature of 30° C. at pH 4 by the addition of phosphoric acid.

The initial solution had no hydrolyzed phospholipids, while the final suspension after 24 hours had a reaction yield in GPI of 80%.

### Example 2

Suspension Microfiltration with Liposomes from Enzymatic Hydrolysis of Sunflower Lecithin

200 liters of a suspension obtained by enzymatic hydrolysis of sunflower lecithin through phospholipase enzymes PLA1 and PLA2 (EXAMPLE 1) are microfiltered in a tangential microfiltration system with sintered steel membrane with a porosity of 0.1 microns. The temperature is maintained at 30° C. and the pressure in a range from 2.0 to 2.5 bar. A recirculation is obtained containing unreacted lipids and phospholipids, free fatty acids and water and a clear permeate containing glycerophospholipids, glycerol, enzyme, salts and free fatty acids.

### Example 3 (Comparative)

Ultrafiltration of Microfiltered Solution from Enzymatic Hydrolysis with Cut-Off 5 kDa

200 liters of clear solution microfiltered at 0.1 micron containing 3 g/l of GPI are filtered with a 4040 membrane in PES of 5 kDa of molecular cut, at a pressure of 6.5 bar and at a temperature of 25° C. and the permeate and recirculation are collected separately.

Surprisingly and contrary to expectations, the permeate contains a low concentration of GPI (0.2 g/l) while the recirculation concentrates in GPI, with a final situation of a ratio of permeate volumes 150 liters and recirculation 50 liters and concentration of GPI in final recirculation of 11.2 g/l.

### Example 4

Ultrafiltration at 50 kDa of Microfiltered Solution from Enzymatic Hydrolysis

200 liters of clear solution microfiltered at 0.1 micron containing 3 g/l of GPI are filtered with a 4040 PVDF membrane of 50 kDa of molecular cut, at a pressure of 3.0 bar and at a temperature of 25° C. and the permeate and recirculation are collected separately.

The overall collected permeate (150 litres) has a GPI concentration of 2.9 g/l and the recirculation has a GPI concentration of 3.0 g/l. The recirculation is then diluted 1:1 v/v with deionized water and filtered again; the permeate obtained has a GPI concentration of 1.5 g/l, the same for recirculation (1.5 g/l).

### Example 5

### Second Ultrafiltration at 5 kDa of Solution Ultrafiltered at 50 kDa

200 liters of clear solution coming from a 50 kDa ultrafiltration permeate containing 2.5 g/l of GPI (Example 4) are filtered with a 4040 membrane in PES of 5 kDa molecular cut, at a pressure of 6.0 bar and at a temperature of 25° C. and the permeate and the recirculation are collected separately.

The permeate has a concentration of 2.3 g/l of GPI while the recirculation maintains a concentration of 2.5-2.6 g/l of GPI during permeation; the recirculation volume obtained at the end of filtration (40 litres) is diluted 1:1 v/v with deionized water and filtered again at 5 kDa under the same conditions as the previous filtration (6.0 bar and T=25° C.), obtaining 40 liters of permeate at 1.3 g/l and 40 liters of recirculation at 1.3 g/l in GPI.

### Example 6

### Nanofiltration at 150-300 Da of Ultrafiltered Solution at 5000 Da

200 liters of clear solution coming from an ultrafiltered solution containing 1.5 g/l of GPI (Example 5) are filtered with a 4040 membrane with 150-300 Da of molecular cutting, at a pressure of 35.0 bar and at a temperature of 25° C. and the permeate and recirculation are collected separately.

The final recirculation (40 litres) has a GPI concentration of 7.2 g/l while the permeate has a GPI concentration <0.05 g/l.

### Example 7

### Electrodialysis

The fluid coming from the membrane filtration process (example 6) is treated in an electrodialysis system preferably in the AEM-CEM-BPM, or AEM-BPM configuration. The fluid generally has a GPI concentration of 0.5-5.0 g/l; the product is processed and the GPI concentrated X2-X4, while it is partially separated from the uncharged components and from GPC and GPE. The % recovery of the GPI appears to be 70-90%.

### Example 8

### Ion Exchange Chromatography

The product obtained from example 7 is subject to ion exchange chromatography as previously described, to give pure GPI.

The purity obtained from the above process expressed in GPI-H or in GPI-X (where X is the cation that neutralizes the GPI-anion) is >95% w/w.

### Biological Experiments

The purpose of the experiments conducted is the in vitro evaluation of the anti-inflammatory activity and of the immune modulating power in the response to bacterial or viral activation, of monocytes activated and treated with GPI. Measurements of protein production or miRNA levels were expressed as an increase relative to the respective control (unstimulated) cells.

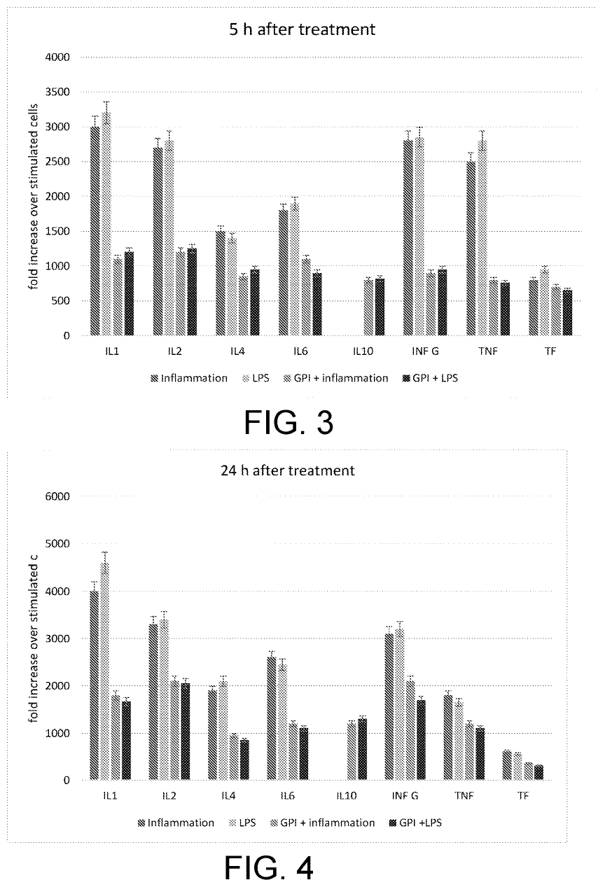

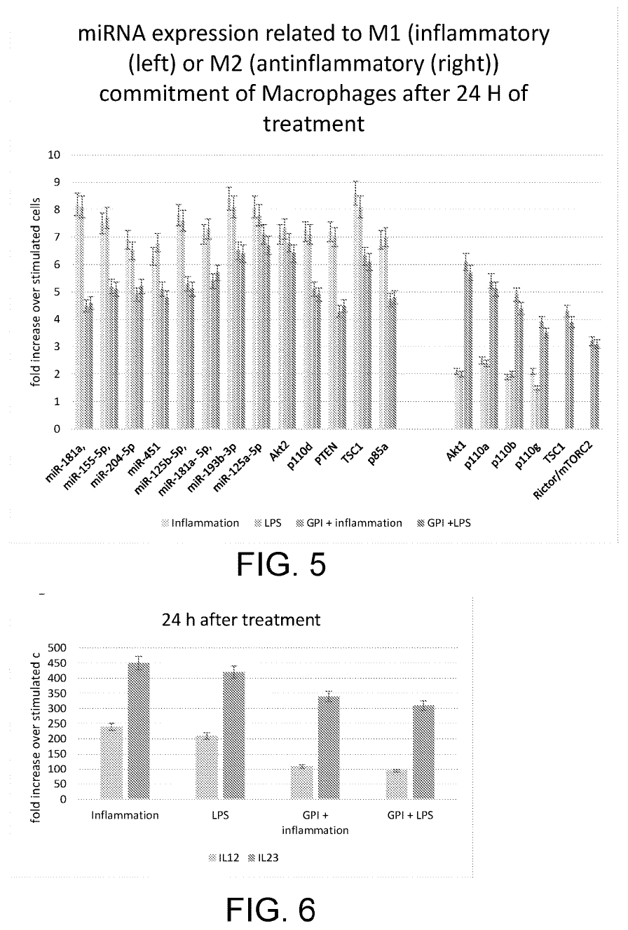

The inhibitory effects of GPI are visible at the level of protein expression after 5 hours (FIG. 3) and 24 hours (FIG. 4) of treatment and at the miRNA level after 24 hours of treatment (FIG. 5).

As regards the anti-viral action, this was evaluated by paying attention to interleukins (IL) 12 and 23. The family of interleukins (IL)-12 is composed of pro- and anti-inflammatory cytokines which are able to signal activation of host antiviral immunity while preventing hyper-reactive immune reactions due to active virus replication and viral clearance. IL-12 and IL-23 are produced and released by immune cells such as monocytes and macrophages to signal T-cell proliferation and release of effector cytokines, which subsequently activate host defense against viral infections (1-8).

Our results (FIG. 6) confirm that monocytes, once activated and treated with GPI, show a reduction of these ILs after GPI treatment.

### Monocytes

Human monocytes were isolated from fresh buffy coats of healthy blood donors, kindly provided by the Centro Immunotrasfusionale, Hospital of Ferrara, Italy, through a Ficoll-Paque™ PLUS gradient followed by Percoll™. PBMCs were firstly obtained by 1.077 g/ML Ficoll-Paque™ PLUS (GE Healthcare Life Sciences, Little Chalfont, UK) density gradient separation according to the manufacturer's protocol. After collecting the mononuclear cells located at the interface between Ficoll and plasma-medium layer, these were washed with PBS, then layered over a solution of 1.130 g/mL Percoll™ (GE Healthcare Life Sciences) before centrifugation at 1650 rpm for 30 min at 4° C. The monocyte layer, which appeared cloudy and occasionally with few red spots, was carefully aspirated and washed with PBS to remove traces of Percoll™. The harvested monocytes were then suspended in RPMI 1640 medium (EuroClone) containing 2% FBS, and incubated at 37° C. and 5% CO₂. After 1 h, non-adherent cells including residual lymphocytes were removed, and monocytes were detached using a cell scraper, then seeded onto plastic culture wells.

To simulate an inflammatory environment in vitro, cDMEM was supplemented with 10 ng/mL Recombinant Human IL-1β, (PeproTech, London, UK) and 10 ng/mL Recombinant Human TNF-α (PeproTech), generating the inflammatory-cDMEM (i-cDMEM).

To stimulate a bacterial like environment in vitro, cDMEM was supplemented with 0.1 ug/ml of LPS (Sigma).

Treatment with GPI: 300 uM.

### EELISA Test Assays

The supernatants of the cultures after 5 and 24 h were collected, centrifuged at 400×g for 10 min at 4° C., then analyzed for TF HGF, PGE-2 and IL-10 production using commercial ELISA kits (Thermo Fisher Scientific) according to the manufacturer's instructions.

RNA Extraction and First-Strand cDNA Synthesis

Total RNA was extracted from cultures after 12 and 24 h using the total RNA purification Plus kit, The RNA quality and concentration of the samples were measured with the NanoDrop™ ND-1000 (Thermo Fisher Scientific). For each sample, 500 ng of total RNA was reverse-transcribed using an RT² First Strand kit (Qiagen, Hilden, Germany) in a final reaction volume of 20 μL.

### Real-Time PCR

Real-time PCR was performed according to the user's manual of the Inflammatory RT² profiler PCR Array (Qiagen) with a StepOnePlus™ Real-Time PCR System (Applied Biosystems™, Foster City, CA, USA) and using RT² SYBR Green ROX FAST Master Mix (Qiagen). Thermal cycling and fluorescence detection were as follows: 95° C. for 10 min, followed by 40 cycles of 95° C. for 15 s, and 60° C. for 1 min. At the end of each run, a melting curve analysis was performed using the following program: 95° C. for 1 min, 65° C. for 2 min with optics off, 65° C. to 95° C. at 2° C./min with optics on. Data were analyzed using the Web-based PCR array data analysis software, which is available at https://www.qiagen.com/it/shop/genes-and-pathways/data-analysis-center-overview-page/.

### Statistical Analysis

All results are expressed as the mean±standard deviation (SD) obtained from at least three independent experiments. Significant differences among groups were determined by analysis of variance (ANOVA). Student's t-test was performed to determine the statistical significance between two samples. Different labels indicate *p<0.05, **p<0.01, and ***p<0.001.

### Results

GPI reduce inflammatory cytokine production.

We cultured monocytes in:
an environment that mimic inflammation in absence or presence of GPI at a concentration of 300 uM.
an environment that mimics sepsis with LPS in presence or absence of GPI at a concentration of 300 uM.
an environment that mimics viral infection in presence or absence of GPI at a concentration of 300 uM.

### we Tested:

Inflammatory cytokyne production: IL1, IL2, IL4, IL6, INF G, TNF alfa, TF
Anti inflammatory cytokine production: IL10
miRNA related to M1 (inflammatory) phenotype: miR-181a, miR-155-5p, miR-204-5p, miR-451, miR-125b-5p, miR-181a-5p, miR-193b-3p, miR-125a-5p, Akt2, p110d, PTEN, TSC1, p85a
miRNA related to M2 (anti inflammatory) phenotype: Akt1, p110a, p110b, p110g, TSC1, Rictor/mTORC2
IL related to viral infection: IL12 and IL23 are activated in presence of viral infection.

Measurements of protein production or miRNA levels were expressed as fold increase over the respective control (unstimulated) cells, and are means (SD) of triplicates from one representative experiment of 4 experiments performed with cells from different donors.

The inhibitory effects of GPI are evident at the protein expression level after 5 hours (FIG. 3) and 24 hours (FIG. 5) of treatment and at miRNA level after 24 h of treatment (FIG. 5).

The interleukin (IL)-12 family consists of pro- and anti-inflammatory cytokines that are able to signal the activation of host antiviral immunity while preventing over-reactive immune reactions due to active virus replication and viral clearance. Amongst others, IL-12 and IL-23 are produced and released by innate immune cells such as monocytes and macrophages to signal the proliferation of T cells and release of effector cytokines, which subsequently activate host defence against virus infections. To this view we search the production of IL12 and 23 during inflammation. Our result (FIG. 6) confirm a reduction of these IL after treatment with GPI.

### Pharmaceutical Formulations

The formulations according to the invention can be prepared according to conventional methods, such as those described in Remington's Pharmaceutical Sciences Handbook, Mack Pub. Co., N.Y., USA, 17th edition, 1985 or in Remington, The Science and Practice of Pharmacy, Edited by Allen, Loyd V., Jr, 22nd edition, 2012.

The invention will now be further described through the following formulation examples.

### FORMULATION EXAMPLES

### Example 1—Tablet

| | | |
|---|---|---|
| GPI | 100 | mg |
| Microcrystalline cellulose | 160 | mg |
| Starch | 28 | mg |
| Lactose | 100 | mg |
| Stearic acid | 6.0 | mg |

### Example 2—Injectable Formulation

Vial 1

| | | |
|---|---|---|
| GPI | 50 | mg |

Vial 2

| | | |
|---|---|---|
| Dibasic sodium phosphate•12H₂O | 12 | mg |
| Monobasic potassium phosphate•2H₂O | 1 | mg |
| Sodium chloride | 32 | mg |

| | |
|---|---|
| Water for injections | to 4 ml. |

1. A process for preparing glycerophosphoinositol from crude or partially purified phospholipid mixtures, comprising the following steps in sequence:
a) hydrolysis of a crude or partially purified phospholipid mixture by treatment with PLA1 and PLA2 enzymes;
b) microfiltration of the mixture from step a) to obtain a microfiltrate, and subsequent ultrafiltration and nanofiltration of the microfiltrate to give a concentrated aqueous fraction of reaction products;
c) electrodialysis of the aqueous fraction of step b) for separating ionic compounds from neutral compounds;
d) ion exchange chromatography.

2. The process according to claim 1, wherein the crude or partially purified phospholipid mixture of step a) has a phospholipid concentration in water between 10 and 100 g/kg of mixture.

3. The process according to claim 2, wherein the phospholipid mixture is a fine dispersion of lecithin in water, wherein lecithin has a concentration between 45 and 55 g/kg.

4. The process according to claim 1, wherein an amount of PLA1 and PLA2 enzyme in step a) is in a range between 200 and 4000 mg/kg of mixture.

5. The process according to claim 4, wherein step a) is performed with a mixture of PLA1 and PLA2, wherein a concentration of PLA1 and a concentration of PLA2 are between 800 and 1300 mg/kg of aqueous suspension.

6. The process according to claim 1, wherein step a) is performed at a pH between 3.5 and 5.5 and at a temperature between 15° C. and 55° C., and wherein an initial pH of the reaction mixture of about 6.5 is brought to a range of pH=3.5-5 by addition of concentrated phosphoric acid.

7. The process according to claim 1, wherein in step b) the pH is adjusted to about 7 by addition of a base comprising a concentrated NaOH or KOH solution.

8. The process according to claim 1, wherein in step b) the microfiltration is performed by tangential filtration on polymer membranes, ceramic membranes, or steel membranes having a porosity between 0.45 and 0.1 micron, and wherein the microfiltration is followed in sequence by a diafiltration.

9. The process according to claim 1, comprising a first ultrafiltration and, a second ultrafiltration, wherein the first ultrafiltration and the second ultrafiltration are performed on wound spiral polymer membranes, and wherein:
the first ultrafiltration is made using membranes with a cut-off in the range from 20 kDa to 500 kDa,
the second ultrafiltration is made using membranes with a cut-off in the range from 1 kDa to 10 kDa,
and wherein the nanofiltration is performed on wound spiral polymer membranes, with a cut-off value ranging from 150 Da to 3000 Da.

10. The process according to claim 9, wherein step b) comprises the following sub-steps:
b1) ultrafiltration of the microfiltration product with a wound spiral polymer PES membrane with a cut-off value of about 50,000 Da;
b2) subsequent ultrafiltration with a membrane having a cut-off of about 5000 Da, wherein GPI, GPE, GPC and other molecules with low molecular weight permeate and further oligomers recirculate;
b3) membrane nanofiltration with a cut-off value of about 1000-150 Da, with a wound spiral PES membrane, to obtain a partial separation between GPI, kept under recirculation, and GPC and GPE passing with the permeate;
b4) diafiltration with demineralized water with low conductivity to obtain a recirculation solution mainly containing GPI and relatively lower concentrations of GPC and GPE with respect to the fraction of step b3).

11. The process according to claim 1, wherein in step c) an aqueous solution is circulated through an electrodialysis system, to obtain migration of the GPI salts and the preservation of GPC and GPE in a main flow solution.

12. The process according to claim 1, wherein the permeated solution is further subjected to a second step of nanofiltration with membranes in the range from 1000 Da to 150 Da to eliminate mainly inorganic substances with low molecular weight and to concentrate the solution.

13. The process according to claim 1, wherein step d) of ion exchange chromatography comprises i) preliminary ion exchange on strong cationic resin in H+ form of the aqueous solution containing GPI, GPE and GPC at varying concentrations, and then ii) passage of the eluted solution on anionic resin in strong or weak OH— form, and wherein, if using weak anionic resins, the resin is first filled with solution from the acid exchange, then washed with water, with a diluted solution of formic, acetic or propionic acid at a concentration of about 1-3% w/v to remove less acidic impurities, and then eluted with a solution of formic, acetic or propionic acid at 5-10% w/v to obtain a pure GPI solution.

14. The process according to claim 13, wherein step d) is performed with a strong sulfonic resin in H+ form, followed by a weak resin in OH— form, wherein the weak base resin is first washed with 12 g/l acetic acid and then eluted with ammonium carbonate at a concentration of 0.03 M.

15-18. (canceled)

19. A method for preventing or early treating a syndrome due to COVID-19 or other viral infections, comprising administering an effective amount of glycerophosphoinositol purified as obtained by the process according to claim 1.

20. The method according to claim 15, wherein the syndrome is sepsis due to COVID-19 or other viral infections.
